(19) United States
(12) Patent Application Publication
(10) Pub. No.: US 2020/0393902 A1
(43) Pub. Date: Dec. 17, 2020

(54) WEARABLE CLOSED LOOP AI WITH LIGHT BASED BRAIN SENSING: TECHNOLOGY AT THE BOUNDARY BETWEEN SELF AND ENVIRONS

(71) Applicant: Blueberry X Technologies Inc., Toronto (CA)

(72) Inventors: William Steve G. Mann, Toronto (CA); John David Chibuk, Toronto (CA)

(21) Appl. No.: 16/898,788

(22) Filed: Jun. 11, 2020

Related U.S. Application Data
(60) Provisional application No. 62/859,761, filed on Jun. 11, 2019.
(60) Provisional application No. 62/958,008, filed on Jan. 7, 2020.

(51) Int. Cl. G06F 3/01; A61B 5/16; A61B 5/00; A61B 5/1455; A61B 5/145

(52) U.S. Cl. CPC G06F 3/015; A61B 5/165; A61B 5/486; A61B 5/14551; A61B 5/7455; A61B 5/7264; A61B 5/7445; A61B 5/7405; A61B 5/14546

(57) ABSTRACT

Means, apparatus, and methods of sensor/sensory, meta-sensory, and meta-sensing user-interfaces are provided. In one embodiment, smart headwear senses at least one health or mental health parameter of a wearer of the smart headwear. In one embodiment a smart eyeglass senses brain activity. In another embodiment a wearable device senses blood flow, and indirectly through artificial intelligence, other health parameters such as fever, brain health, mental health, and the like. In another embodiment, a wearable AI (Artificial Intelligence) device has associated with it a meta-lock-in amplifier, i.e. a second lock-in amplifier responsive to an output of a first lock-in amplifier, where the first lock-in amplifier is referenced to at least one alternating current electrical signal driving a light source, and the second lock-in amplifier is referenced to an output of the first lock-in amplifier. In another embodiment, a collective of users engage in a gamelike activity that promotes improved physical and mental health. When paired with a camera a wearer can automatically capture rushing and dragging moments during their day as blood rushes or drags or maintains tempo in their brain. When paired with a fuzzy display a wearer can gain insight in real-time to their cognitive state.

This application claims the benefit of earlier filed provisional application U.S. Provisional Patent no. 62/859,761 filed Jun. 11, 2019 the entirety of which is incorporated herein by reference off which the following is a specification:

### FIELD OF THE INVENTION

The present invention pertains generally to a new kind of input/output human sensory interface device that may be used to monitor or improve physical or mental health. In one of its aspects, the present disclosure relates generally to U.S. Provisional Patent no. 62/859,761 discloses a system for monitoring and modification of the cognitive state of a human through a head-worn real-time hemodynamic measurement and feedback device.

### BACKGROUND OF THE INVENTION

There is a growing need for physical and mental health, wellbeing, wellness, and fitness. For example, the growing population of the elderly need a way to stay fit and healthy, and everyone will welcome wearable AI that can create a foundation for improved well-being. Another example is a child that suffers from attention deficit disorder that needs a way to manage their mental health.

This generally relates to the combination of light based brain sensors, feedback stimulus, displays and secondary sensors including a camera to provide feedback in a closed loop manner to a wearer as they interact with themselves, machines and their environment.

### SUMMARY OF THE INVENTION

The following briefly describes a new invention. It is possible with this invention to provide a health-sensing apparatus as well as an environment-sensing apparatus in conjunction with a system that functions as a true extension of the human mind and body.

In one embodiment an eyeglass-based apparatus monitors brain activity and provides feed-back to create a HI (humanistic intellingence) feedback loop. In one embodiment an autodarkening eyeglass provides brain-based darkening, in response to SSVEP visual information.

In another embodiment an eyeglass having a soft strap is suitable for sleeping and provides darkening as one falls asleep, or in the morning in response to morning light, allowing the light in when it is time to wake up, but blocking the light before such time. The darkening may also be adjusted during a period of high stress and or low focus.

In another ebodiment there is provided time domain or functional NIRS (functional near-infrared spectroscopy) feedback to capture blood oxygenation changes in brain tissue to capture periods of fatigue, high engagement, mental stress, audio, motor and visual response for improved effectiveness and improved mental health.

In another ebodiment there is provided EOG (electro oculo gram) feedback so as to help capture lucid dreams and the like, in an interactive virtual reality environment for improved effectiveness and improved mental health.

In another ebodiment a left and or right side fNIRS feedback to capture ocular blood changes in eye tissue to measure eye movement.

In another ebodiment a photodiode sensing feedback loop to control the maximum and minimum brightness of a feedback LED or electronic tinting display.

The invention may take the form of an eyeglass with one or two eyeglass lenses, i.e. one lens for both eyes, or separate lenses. It may also take the form of a clip on device that slides onto a temple side piece of an eyeglass device. It may thus embody a prescription or be separate from it.

In one embodiment, a fractal mesh-based brain-computer interface is constructed as a skull cap that has the architectural form of a neural network or neurons, i.e. a master node that branches to two sub nodes that each branch to two more, and so on. At each level there are more nodes but they are smaller and less reliable. So we have a small number of highly reliable nodes, as well as a large number of less reliable nodes, and so on, but the nodes are redundant, and therefore contribute to a machine learning algorithm that learns and understands the brain and forms thus a kind of informatic “exoskeleton” for the brain, thus forming an advanced adaptive interface. This “mindmesh” is useful in a wide variety of interfaces.

In another embodiment, the fractal mindmesh takes form in a bicycle helmet.

In one embodiment for adaptive human-powered transportation, a bicycle with sensors and meta-sensors results in a cybernetic control system in which aspects of the bicycle are responsive to the mental and physical state of a cyclist, as well as the environment around the bicycle and cyclist.

In one embodiment there is an electric machine in the bicycle together with a transmission, both of which are responsive to the cyclist and environment.

In another embodiment, there is are a plurality of electric machines.

In some embodiments, there is included an electric machinette, i.e. a small electric machine not designed or intended to generate or receive substantial electric power, but, rather, intended as a sensor for sensing rotation and related phenomena.

In other embodiments, there is an electric machine in a mobility device that is responsive to the mental and physical state of a rider.

In one embodiment there is an electric machine and means for human power input, and means for optimally combining the human power input with a power input or output (e.g. dynamic braking) of an electric machine, where the power distribution is responsive to sensors that sense a mental or physical state of a user, and sensors that sense a state of the environment around the user.

In one embodiment there is an electric mobility device that learns from observation of its environment.

In another embodiment there is a collective of mobility device users who help each other navigate by automatic generation of accessiblity maps.

In another embodiment there is a legitimacy exchange for sensors using machine learning, AI (Artificial Intelligence) and blockchain authentication of shared sensory maps.

In another embodiment there is a smart building responsive to a wearable device which is also responsive to the building.

In another embodiment there is a smart building responsive to a plurality of the wearable devices, which are responsive to the building.

In another embodiment there is a smart city responsive to a wearable device which is responsive to the smart city.

In another embodiment there is a mind-controlled device that is responsive to the human mind and a smart vehicle that is responsive to the wearable device, and a smart city that is responsive to the smart vehicle, and vice-versa.

The following provides an informal review/summary of the new invention.

The invention facilitates sensing and meta-sensing for adaptive human interfaces to head-worn technology and the like, which may also be a means to connect to and interface to various other devices such as human-powered, or partially human-powered transportation within the context of smart cities, or with smart buildings.

In one aspect the device can be or facilitate cyborg (cybernetic organism) feedback, and embody HI (Humanistic Intelligence).

One aspect of the invention helps a person interact with a device such as a machine, computer, smart room, smart office, smart tablet, smart phone, smart vehicle, smart city, or the like.

One aspect of the invention allows a bicycle rider to sense the world and affect the world through cybernetic biofeedback.

One aspect of the invention creates an accessibility map of the world in regards to shared sensory fusion.

Another aspect of the invention uses a collective of users to gather data about the world, roadways, sidewalks, as well as indoor spaces, and share that data in service of sight, to assist other users with mobility, as well as to assist visually impaired users with mobility.

Another aspect of the invention uses meditation and mindfulness to help a person engage their own physical mind and body more effectively.

Another aspect of the invention uses collective meditation and mindfulness to help people engage their own physical minds and bodies collectively.

The apparatus of the invention allows the user to both receive and effortlessly convey navigational information as they move through space.

According to one aspect of the invention, there is provided a headworn human-user interface, wherein there comprises one or more light based and or electrical brain sensing units, a processing unit, a memory unit, one or more feedback units and one wireless communication unit.

According to another aspect of the invention, there is provided a wearable camera in a headworn device.

Additional aspects and advantages of the invention will be set forth in what follows.

### BRIEF DESCRIPTION OF THE DRAWINGS

The invention will now be described in more detail, by way of examples which in no way are meant to limit the scope of the invention, but, rather, these examples will serve to illustrate the invention with reference to the accompanying drawings, in which:

FIG. 1 illustrates an inter-connected closed feedback loop whereby a human 110 with a mind 120 and body 140 has afferent 130 and efferent 131 feedback to a machine 160 that has observatibility 150 and controlability 151, the human and the machine are combined 180 which encompasses a cyborg 199 which interacts with a smart city 190 through sousveillance 180 and surveillance 181 by the city

FIG. 2a illustrates the combination of a signal generator 200 to control a series of light emitting diodes “LEDs” 220, 230 and or a second pair 240 with wavelengths that measure target oxygenated, deoxygenated or total hemoglobin states for example 880 nm and 660 nm. A photodiode sensor 230 is used to measure the reflective of light and fed into a lock-in-amplifier 250 which has an input of a trans-impedence amplifier 240 to amplifier the signal into a measurable output from the photodiode. One or more LED pairs 221, 222, 223, driven by one or more transimpedance amplifiers 211, 212, 213, combined represented as 210

FIG. 2b illustrates the combination of L.I.A.=Lock-In-Amplifier 270 time-domain near infrared spectoscopy (TD-NIRS), photoplethysmogram (PPG) or Hemoencephalograhy (HEG) sensing sensors using a phase delay intersection 280

FIG. 2c illustrates to combination of one or more outputs from FIG. 2b with time domain delay 291 into a mixerator 292 to combine the signals to generate a complex-valued signal return

FIG. 2d illustrates to combination of two or more signal generators 293 with pairs of light emitting diodes which capture blood flow changes in the oxygenated and deoxygenated light spectrum 294 measured by a photodiode 295 with measured signal input into a demultiplexer and lock-in amplifier 296 to determine a complex-valued signal return change between the two sensing locations the output can be used to estimate pulse transit time

FIG. 3 illustrates a closed loop feedback system to phase coherent feedback through a light based display 350 of a computed complex-valued signal return 340 of light based brain activity whereby the phase 310 is modulated by the feedback of the light emitting diode(s) simultaneously presented to an input brain activity signal through a computation step 320 and compared 330 with a secondary brain activity signal 360, where by the color rotates through the color wheel at a rate relative to phase and the the intensity of light changes relative to the amplitude as represented by 370

FIG. 4 illustrates a phase shift output from two near infrared spectroscopy blood flow sensors 410 and 420 in the complex-valued signal return 430 from a mixerator, the output results in an estimate for pulse transit time relative to phase and amplitude change

FIG. 5 illustrates a closed loop feedback system to determine whether to apply feedback until a mental target state is achieved using up-chirps, steady feedback, down-chirps or time adjusted frequency pulsations from a model applied to a selected feedback stimulus

FIG. 6 illustrates the process for a brain activity sensor and a camera to automatically capture photos or videos as an adjusted frame rate based on the brain activity rushing, in tempo or dragging

FIG. 7 illustrates the process of a brain activity sensor, a camera sensor and feedback stimulus to regulate a person's mental state based on their environment and their measured brain activity by using a feedback stimulus

FIG. 8 illustrates a capacitive touch input 810 that enables a wearer to recenter to a neutral maintained state or slide forward 820 from home to increase a previously software enabled feedback method to a desired level for a pre-determined period of stimulus time for example, 5 seconds, 7 seconds, 15 seconds, 30 seconds, 1 minute, 5 minutes, 10 minutes, 20 minutes

FIG. 9 illustrates a LED 910 or series of LEDs in an array to provide continuous or directed feedback to the wearer through pre-determined software changes of LED colour into discrete or continuous wave state of pulsations or frequency changes

FIG. 10 illustrates a light emitting source 1020 mounted on a printed circuit board 1040 in the form of at least one LED through a fiber optic channel 1010 and bent or refracted through a reflective panel 1030 whereby the angle refracts the light through 1050 an indexed glass or plastic to be displayed on a piece of glass or plastic 1060 mounted on or within an eyeglass

FIG. 11 illustrates a display which outlines shapes and or combinations of shapes as represented by a color, using a color filter different shapes are projected into the field of view of a wearer. Whereby 1110 is the light emitting source and 1120 is the defined color filters

FIG. 12 illustrates a display which is emitted through a light emitting source 1210 through a laser-induced porous graphene films from commercial polymers 1230 to demonstrate icons for actions for the wearer to take such as but not limited to walking, breathing, eating, meditating, water. The display may also include a analog or digital clock 1220

FIG. 13 illustrates the regions of brain for measurement for an eyeglass form, whereby 1310 denotes the brocca region of the brain, 1320 denotes the inferior frontal gyrus region, 1330 the anterior temporal lobe region, 1340 the temporal lobe and 1350 the temporoparietal junction

FIG. 14 illustrates the various placements of LEDs and photodectors for determining a hemo-dynamic response from brain tissue, whereby 1410 denotes a light emitting source, 1420 denotes a photodetector and 1430 depicts the ratio of distance from the light emitting source to the photodetector

FIG. 15 illustrates a neural colour based encoding sequence to result in a stimulus response where by 1510 is the light emitting diode 1520 is the first color displayed in sequence 1530 is the time between changing the color, 1540 is the second color, the sequence continues for any nth number changes and time intervals, the feedback of the states 1520, 1530 and [1540] may be analog or digital in response intervals resulting in a frequency pulse, chirplet response, chirp-up or chirp-down response

FIG. 16 illustrates the location of one 1610 blood flow measurement sensor locations and 1620 denotes the location of additional sensing locations by additional measurement sensors including blood flow, accelerometer, gyroscope, magnetometer, electrooculography, galvanic skin resistance, camera, barometer, temperature, one, a combination of or all can be included in each of the denoted locations

FIG. 17 illustrates the process of capturing brain activity and audio simultaneously to categorize a response automatically from understanding brain activity as it relates to audio and presenting a feedback output to the wearer

FIG. 18 illustrates the process of filtering, segmenting and classifying a brain activity sensor to be stored or whether to apply feedback

FIG. 19 illustrates the flow of a brain activity and a generated token or blockchain of value based on brain activity to be exchanged by a 3rd party

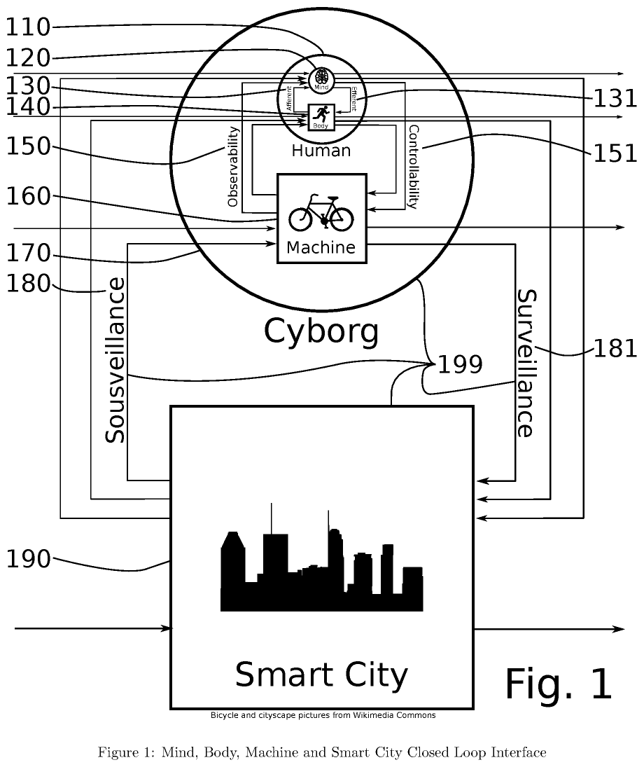

### DETAILED DESCRIPTION OF THE PREFERRED EMBODIMENTS

While the invention shall now be described with reference to the preferred embodiments shown in the drawings, it should be understood that the intention is not to limit the invention only to the particular embodiments shown but rather to cover all alterations, modifications and equivalent arrangements possible within the scope of appended claims.

In all aspects of the present invention, references to “camera” or “detector” mean any device or collection of devices capable of simultaneously determining a quantity of light arriving from a plurality of directions and or at a plurality of locations, or determining some other attribute of light arriving from a plurality of directions and or at a plurality of locations.

References to “processor” , or “computer” shall include sequential instruction, parallel instruction, and special purpose architectures such as digital signal processing hardware, Field Programmable Gate Arrays (FPGAs), programmable logic devices, as well as analog signal processing devices.

When it is said that object “A” is “borne” by object “B”, this shall include the possibilities that A is attached to B, that A is part of B, that A is built into B, or that A is B.

When it is said that an object is a a “temporalizer” where temporalizer is a general and/or phase shifter for example delay, phase shift, amplitude shift, when it is said an object is a compardjustor comparametrically adjust in quantum field theory amplitude domain. A generalized mixing function; i.e. the law of composition. When it is said a mixerator be a general mixer where there is some Mixer. When it is said a mental pitch pipe is a frequency of signal to which blood oxygenation in brain tissue responds to stimulus. When it is said a mind pattern-er is a pattern that is produced by brain activation and is matched to another brain activation pattern. When it is said a flow profile is a profile of mental state for an individual or group of individuals. When it is said a mental target is a target mental state. When it is said “rushing” is when blood rushes to a region of a person's mind. When it is said “dragging” is when blood slowly moves through a region of a person's mind. When it is said “tempo” is when blood flows rhythmically through a region of a person's mind. When it is said “leading” a persons mind is leading infront of another reference signal. When it is said “lagging” a persons mind is lagging behind of another reference signal. When it is said leadback the signal is leading from the back and lagback the signal is lagging from the back. When it is said leadforward the signal is leading from the front and the lagforward when the signal is lagging from the front. When it is said “memory map” is when camera, location, brain activity and sensor information is displaying in time sequence on a map or in a report.

A human 110 has a brain 120 which receives signals over afferent nerve signal path 130 and efferent nerve signal path 131. The efferent nerves receive input from a body 140 to brain 110. Signal paths 130 and 131 facilitate a feedback loop between the brain and the body. The efferent nerves receive input from brain 110 and carry these electrical signals to body 140.

The brain 110 is represented schematically as a circle, and the body, which is a machine of sorts, is represented schematically as a square. The human 110 observes its surroundings by its capacity for obervability, through observability signal flow path 150. The human 110 affects its surroundings by its capacity for controllability, through controllability signal flow path 151. Signal flow paths 150 and 151 establish a feedback loop with machine 160. The human 110 and machine 160 together with the associated feedback loops form a cybernetic organisim, cyborg 170.

Cyborg 170 senses its surroundings through sousveillance (undersight) by way of signal flow path 180. Cyborg 170 affects its surroundings through surveillance (oversight) by way of signal flow path 181. This may be deliberate such as when interacting with a camera-based interactive video display that watches cyborg 170. An example is when cyborg 170 is a human and bicycle, and the cyborg 170 drives past a speed-sensing radar which indicates the speed.

Signal flow paths 180 and 181 form a closed-loop between one or more cyborgs like cyborg 170 and a smart city 190.

A smart city that has only signal flow path 181 does not serve humanity. Although it may serve the police and help in the creation of a police city or a police state, it fails to create a complete truth. It is like a machine that provides no feedback, or a body lacking an afferent nervous system.

Ideally, therefore, the smart city embraces both veillances (sur and sous),not just one veillance. When both veillances are present, in roughly equal proportion (“equiveillance”) we have a cybernetic smart city 199.

The relationship between the brain and body of the human is mimiced in a relationship between human and machine, to form the cyborg. The relationship between the human and machine is mimiced in the relationship between the cyborg and smart city, to form the cybernetic smart city.

Therefore, we observe a fractal (self-similar) nature in the overall architecture, as well as its components such as the mindmesh which mimics human neural circuitry.

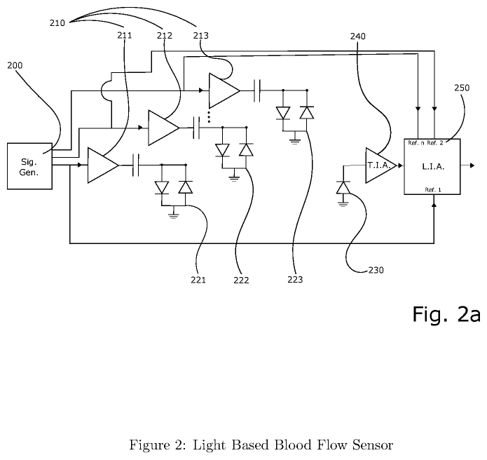

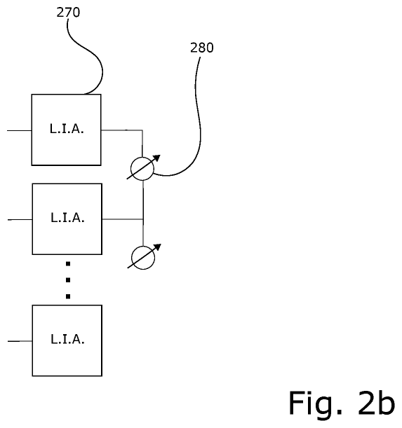

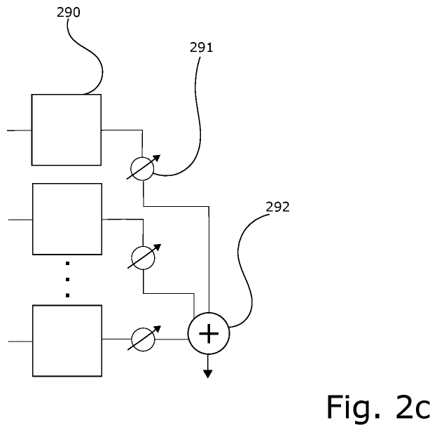

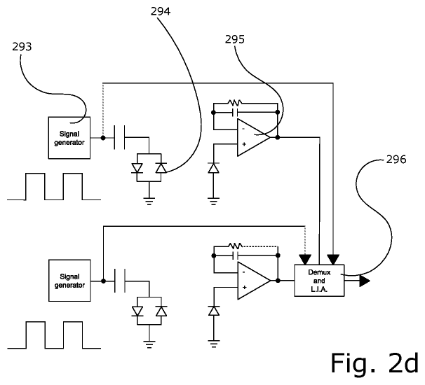

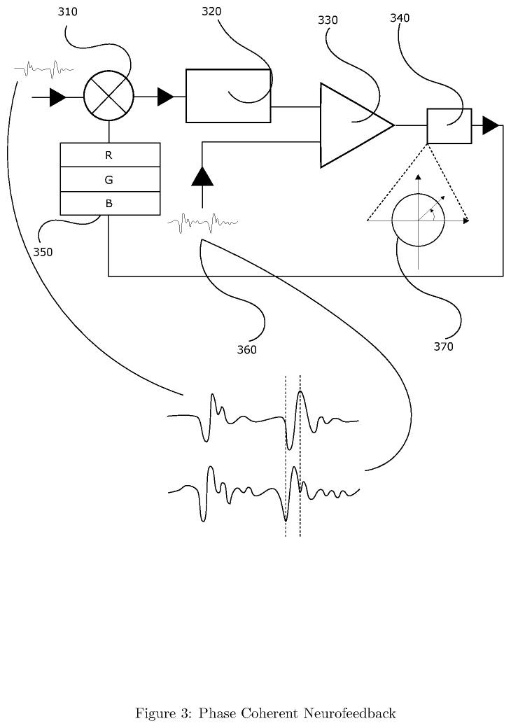

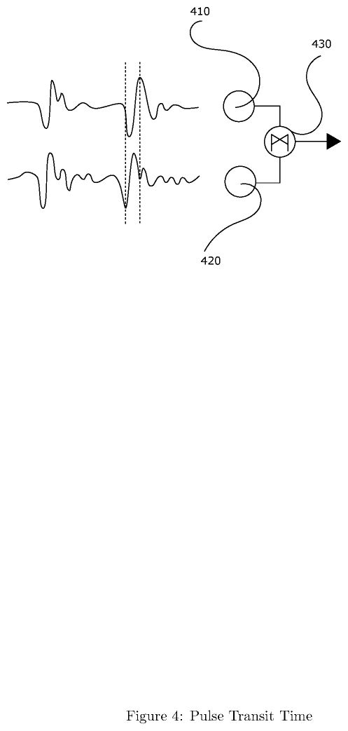

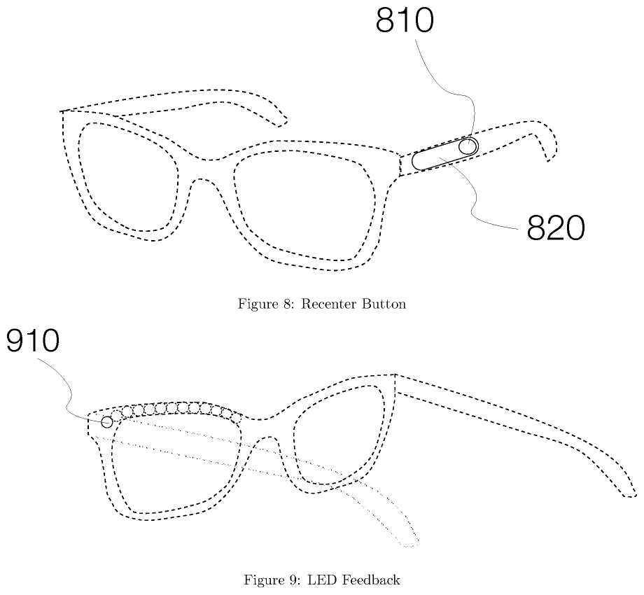

FIG. 2 is a diagram depicting an example of an undigital cybernetic interface based on measuring changes in blood flow in tissue within a smart eyeglass FIG. 8

The smart eyeglass encompasses the field of computer processing of the output of a function near infrared spectroscopy fNIRS, time-domain near infrared spectroscopy “TD-NIRS” or hemoencephalography HEG sensor to provide a hemodynamic response measurement also known as a BOLD response. Specifically placing the hemodynamic sensor at or around the left or right temporal lobe region and or left or right inferior frontal gyrus IFG region, determining the level of activity for specific tasks for either placement on the left or right or both temporal lobe regions and or the left or right prefrontal cortex. The methodologies outlined are used to provide classifications for hemodynamic response, cognitive states, and semantic understanding. The sensor measurement is classified and paired with a feedback mechanism of audio, visual, haptic and or electrical stimulation through direct or alternating current.

Methods to automatically detect brain activity classifications based on defined use cases described below. Wearable devices have detected patterns in heart rate, movement, muscle and brain activity from EEG sensors. A new challenge exists in utilizing a hemodynamic sensor to monitor and classify activity types and levels for the sensor location to the left or right temporal or IFG region of the brain.

In order to detect additional classifications for specific processes for the part of the brain a single or multi-nodal hemodynamic sensing device can be utilized to localize a blood oxygenation level and changes in deoxygenated, oxygenated and total hemoglobin to the part of the brain to determine activity level. The sensor data can be combined with a multi-axis accelerometer, multi-axis gyroscope, a magnetometer, a temperature sensor, a microphone, an ambient light sensor and or a galvanic skin sensor.

The process to determine a hemodynamic response measurement from multiple LEDs and photodiodes ‘detectors’ to determine a change in oxygenated blood oxygenation, deoxygenated and total blood oxygenation change is depicted in FIG. 2. LED1 220 has a wavelength of 500-900 nm, LED2 230 has a wavelength of 800-900 nm, LED3 has a wavelength of 500-700 nm, and LED4 has a wavelength of 900-950 nm. The distance from LED1 to Photodiode 1 is from 5-15 mm, the distance from LED2 to Photodiode 1 is from 5-15 mm, the distance from LED3 to Photodiode 1 is from 5-15 mm, from LED4 to Photodiode 1 is from 5-15 mm, the distance from LED1 to Photodiode 2 is from 15-30 mm, the distance from from LED2 to Photodiode 2 is from 15-30 mm, the distance from from LED3 to Photodiode 2 is from 5-15 mm, the distance from LED4 to Photodiode 2 is from 5-15 mm. LEDs of 240 may contain LED3 and or LED4 in position between PD1 and PD2 and or to the right of PD2 of distance 5-15 mm. A computation step is utilized as a weighted optional measurement from the modified beer lambert law utilized that is known knowledge to determine a change in hemoglobin concentration. The computation step combines extinction coefficient and differential length path factor (DPF) values for the specific LED wavelengths selected. The computation step in computation step may utilize a combination of 2 or more LED and Photodiode pairing measurements to determine a result of change in oxygenated, deoxygenated or total hemoglobin in a single measurement time step. The combination of weighted value and wavelengths attenuated for water, bulk lipids, oxygenated and deoxygenated hemoglobin concentration changes. The distances of the photodiodes result in approximated depth measurements of surface skin layer, cerebral fluid, grey and white matter brain tissue. The computation step takes inputs from the output of 270 to include the mammals age, weight, hair colour, hair thickness, skin colour, skin roughness, sensor location.

In the context of wearable computing a simplified and wearable form factor is needed to address this need. As depicted in FIG. 8

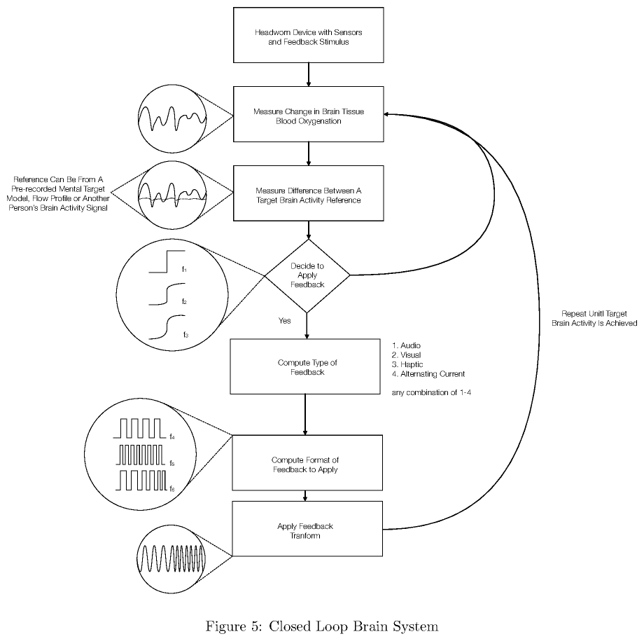

Using a closed system feedback loop as depicted in FIG. 5 by measuring the hemodynamic response of a wearer in the temporal or prefrontal cortex location a closed loop model illustrates a current modulation to affect the hemodynamic response in a region of brain tissue, e.g. if amplitude increase or decrease of response occurs could lead to a positive or negative affect on; memory, relaxation and or sleep calmingness to that region of the brain as applied. For example if initial change is 50 percent and is reduced to 20 percent by affect. The same feedback loop can be applied with alternating current, direct current, visual light array stimulus, audio binaural tones and or haptic feedback pulsations. The feedback loop is determined through a “rushing”, “dragging”, “leading”, “lagging”, “leadback” , “lagback” , “leadforward”, “lagforward” frame work where the phase is determine between a measure and reference brain activity signal to determine a complex-valued signal return for feedback.

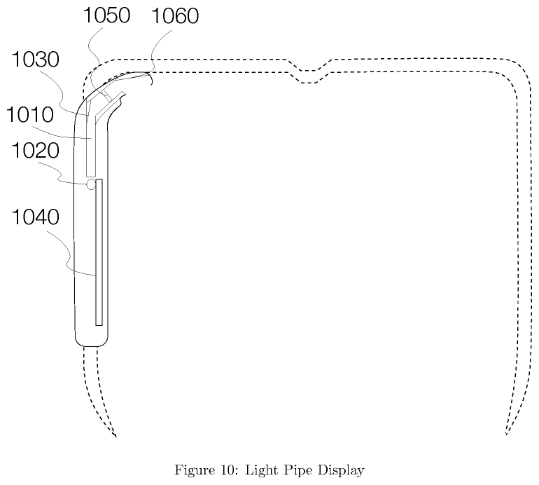

The feedback mechanism to the wearer may include an audio feedback included but not limited to audio cues of binaural frequencies, audio cues of suggesting action or behaviour changes such as fatigue, mental stress, an alternating current, a direct current and or one or series of light emitting diodes as depicted in FIG. 9.

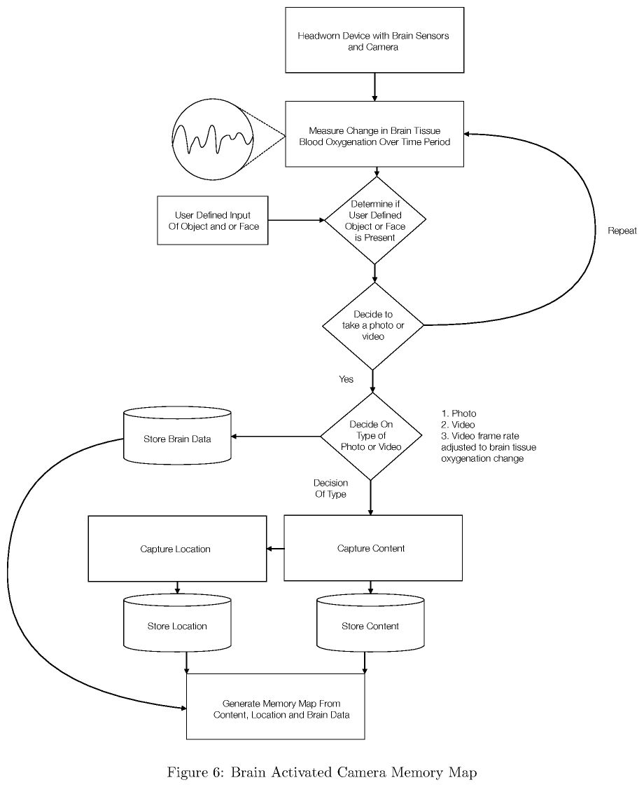

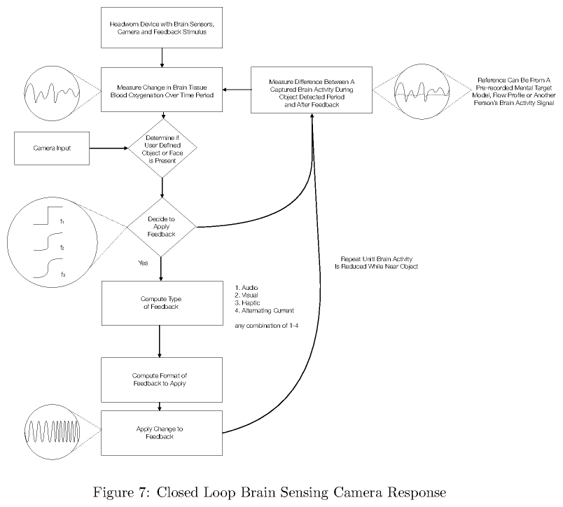

The feedback mechanism to the wearer may include a visual feedback included but not limited to adjustment of a glass lens of colour tint, transparency, focal length and or aperture. The lens in FIG. 16 may adjust using a photochromic electronic current to determine the tint of the lense including but not limited to UV, monochromatic, Lucas2014 Erythropic=5.21 e+3 W/cm2,Lucas2014 Chloropic=5.25 e+13 W/cm2, Bilirubin=778 W/cm2, blue light: 400-480 nm, green light: 480-560 nm, yellow light: 560-580 nm, red light: 580-760 nm. The lens may also be a stacked combination of electronic tint lense for example a red tint, blue tint, green tint and monochromatic tint to result in a combination of light filters and or adaptive LCD gradient states. The feedback technique may also include a liquid crystal display as the eyeglass lens which can change the level of darkness as applied from a current. When the feedback mechanism is a camera FIG. 6 illustrates the flow of a brain activity and camera capture loop for filtering, processing and storage of data to present a memory based log of captured people and objects. When a person has blood rushing to their head they are “rushing” and when there is less blood flowing they are “dragging” . When “rushing” the frame rate of photos or video is increased and when “dragging” the frame rate is decreased in a ladder or analog conversion.

Accordingly there are significant commercial advantages to providing various systems, apparatus and methods to understand brain activity using a hemodynamic sensor placed on the temporal lobe region of the brain. The specific use cases outlined here are as described below.

temporal lobe activity use cases, a list of functions that have been found to change relative to temporal lobe activity and inferior frontal gyrus IFG region activities:

Specifically for right handed individuals in the left hemisphere language processing for phonologic (sounds) and semantic (meaning) and syntax tasks. The following list of activities related to left hemisphere activation:
When learning non-native sounds activity reduced with repetition
Greater reduction predicts better learning
When reordering and comprehending sentences
Long term memory
When recalling semantic (general) facts
Working memory increases in proportion to number of items in working memory during n-back task
Empathy Responds strongly to faces showing emotions
Reducing activity improves and speeds up emotion recognition from faces
Selective attention (reduced) when doing a task and a biological distraction appears (a hand, not moving dots)
If a threatening action seen from a person onto which attention is focused
Cognitive load/task difficulty Increases in proportion to complexity of simulated air-traffic control task (may be interaction with memory)
Activity is reduced with experience for simulated tasks
Inhibiting an initiated response
Cognitive control
Reducing activity relative to right temporal lobe enhances creativity

Specifically for the right hemisphere for right handed individuals, hemispheres may reverse depending on handiness. The following list of activities related to right hemisphere activation:
Memory When recalling autobiographical facts
Semantic memory retrieval inhibiting an initiated response When somebody is asked to do something and then told to stop
Cooperation during a game
Competition during a game
Activity correlated between players brains during competition
Empathy responds to emotive words and faces
Training to increase activity improves accuracy of emotion recognition in faces
Threats activity increases when identifying concealed threats in natural images
Increased activation can improve performance When making difficult/conflicting personal decisions
In more intelligent people it is activated more if choices are harder or more conflicting
Fine motor control can also be interpreted from the right hemisphere.

To detect discernible changes machine learning techniques are applied including pattern matching and automatically generated feature sets from frequency analysis techniques where can be encoded to auto detect activity states of the wearer.

The measurement output of a function near infrared spectroscopy fNIRS or time-domain NIRS “TD-NIRS” or hemoencephalography HEG sensor or photoplethysmogram “PPG” to provide a hemodynamic response measurement also known as a BOLD response which is comprised of a oxygenated and deoxygenated hemoglobin change ratio, with an automatic response in an audio, visual, haptic or electrical response to adjust the cognitive state of the mammal.

Using a closed system feedback loop by measuring the hemodynamic response of a wearer in the temporal location a feedback mechanism can be adjusted to the wearer to increase awareness and adjust the cognitive state.

Where the feedback selection is an auditory, visual, haptic, or electrical stimulation feedback

Where the auditory feedback is a binaural beat of a frequency tone for 10 seconds to 60 seconds

Where the auditory feedback is an adjustment of volume increase and or decrease on the wearers mobile device

Where the visual feedback is a color gradient, bar level displayed through a series of light emitting diodes FIG. 9 or a single light emitting diode to the user on an augmented display and or mobile device

Where the haptic feedback is a short or long pulsation to describe the current measured state

Where the visual feedback is an LED with colors and intensity describing the cognitive level of the wearer and or a photochromic lens or combination of lenses to provide a visual feedback to the wearer adjusting attenuated light through a lens.

Where the effect of the feedback is measured internally by a microcontroller to adjust the next feedback effect

Where the inputs include the hemodynamic response from a fNIRS, TD-fNIRS, PPG and or HEG sensor and a semantic input of text from a written or auditory to text transcription and time.

Where the model utilizes a hidden markov model, neural network and or bayesian network to generate a likelihood function for binary or real number output of cognitive state and or semantic output.

Where the hemodynamic response can be used to predict a semantic vector.

Where the semantic input can be used to predict a cognitive state.

Where the cognitive model taking inputs of [symbol] hemodynamic response, text, time [symbol] provides an output either of [symbol] cognitive state, likelihood [symbol] and or [symbol] cognitive state [symbol] as a fixed classification and or a cognitive state as a real number from 0 to 1.

Where the cognitive model can be take an input of [symbol] cognitive state, hemodynamic response, time [symbol] and provide a likelihood of [symbol] text likelihood [symbol] and or [symbol] text [symbol] as a fixed classification

A computer-implemented method for performing a series of filter, automatic threshold, and model classifications to interpret hemodynamic signals derived from 1 to 3 axials of information provided from the inferior frontal gyrus and or temporal lobe part of the brain. The output of the system provides a specific activity and sub brain activity level in correlation with the task being monitoring. For example when writing a high, moderate and low activity level is determined.

The above described method may require retraining as available population data becomes available from sensor manual data or developed features for separating the raw hemodynamic signal.

The method described above may work on an embedded device to a cloud environment depending on the complexity of the model utilized and desired accuracy output for the selected use case from above.

This method requires input from an hemodynamic sensing device to a) receive data b) filter and extract valuable information c) output a classification and categorization of the provided signal as described below in FIG. 18

A closed loop feedback system to measure the cognitive state of the wearer and provide feedback to adjust the cognitive state.

The above described method may require feedback mechanisms including visual (LEDs, LCDs or graphs), auditory (binaural or volume adjustment), haptic (0.1 to 2 second haptic pulses of sigmoid function intensities from 0.1 to 10 Hz)

The method described above may work on a mammal.

Upon detecting a classification an output is provided to the interface that contains a probability of classification as well as valuable features for the specific task being analyzed.

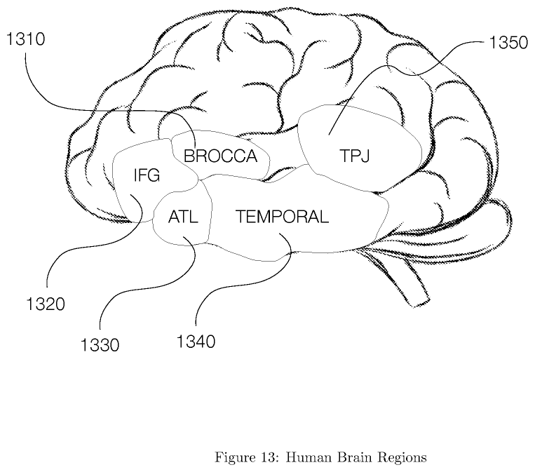

The classification technique is applicable to other regions of the brain including the tempero parietal junction, the prefrontal cortex, brocca region, inferior frontal gyrus region and or the anterior temporal lobe as depicted regions in FIG. 13

A closed loop feedback and prediction system using a measurement including a hemodynamic response and or time and or semantic input to provide a probability output of a cognitive state in real or classification form and or a semantic output with a hemodynamic response, time and cognitive state input.

The above described method may require a baseline model to be used from a generalized dataset to provide a cognitive state probability from an input of one or more input vectors from a single or plurality of sensor inputs containing [symbol] hemodynamic response, time [symbol] or [symbol] deoxygenated hemoglobin change, oxygenated hemoglobin change, time [symbol] or, [symbol] deoxygenated hemoglobin change, oxygenated hemoglobin change, acceleration x, acceleration y, acceleration z, gyroscope x, gyroscope y, gyroscope z, magnitude acceleration, galvanic skin resistance, temperature, time

[symbol]

The method described above may work on a mammal.

The method described above may produce a probability function to transcribe a hemodynamic response to a semantic output.

The method described above may utilize an input of a filtered hemodynamic response, accelerometer, gyroscope, magnetometer, temperature and or galvanic skin resistance.

The output of the classification may also produce an estimated heart rate and heart rate variability measurement.

Embodiments described herein provide a method of classification from a single or plural of hemodynamic sensing devices to determine brain activity classification and sub categorization for a variety of defined use cases as described in [0064] and [0065]

In particular the hemodynamic sensor may be placed on either or both temporal lobe regions, anterior temporal lobe, inferior frontal gyrus, brocca region and or TPJ regions of the brain to determine a specific classification of brain activity data as depicted in FIG. 13 specifically regions inferior frontal gyrus in prefrontal cortex (FT7, FT8, F7, F8) and or left and or right temporal lobe (FT8, FT9, P9, P10, T7, T8) and or the brocca region (C5, C6, FCS, FC6), or the temporoparietal junction (TP7, TP8)

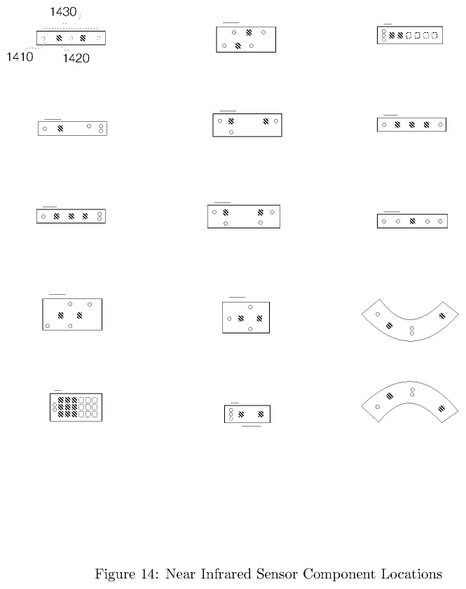

The processing and categorization of the brain sensing data may occur on a microcontroller unit within the hemodynamic device or conducted on a mobile or server based device. The processing method may include conversion of a multiple LED emitter response measurements from multiple photodiodes to convert to a oxygenated, deoxygenated and or total hemoglobin (blood oxygenation) change measurement as depicted in the layout formats within FIG. 14, where by the LEDs are depicted as circles 1410 and photodetectors as squares 1420 and lines represent ratio of distance from LED 1430 to photodetectors the LED wavelengths are described with FIG. 2 illustrates a circuit which results in a measurement output from multiple LEDs and multiple photodiodes and whereby the photodetectors are able to receive a light response between 500 and 1000 nm, the range of the photodetector may exceed this range. This outline potential placements and orientations for combinations of 2 or more photodetectors and 2 or more light emitting diodes to result in the measurement of a hemodynamic response.

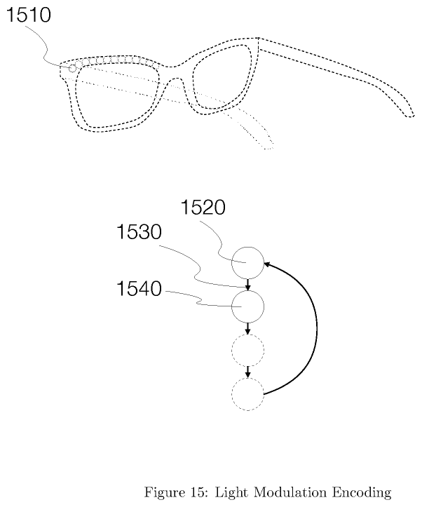

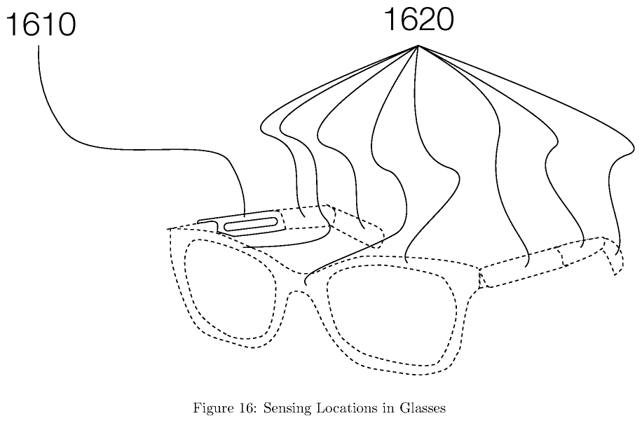

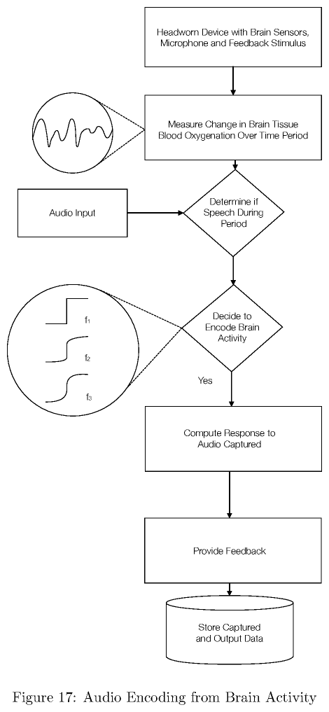

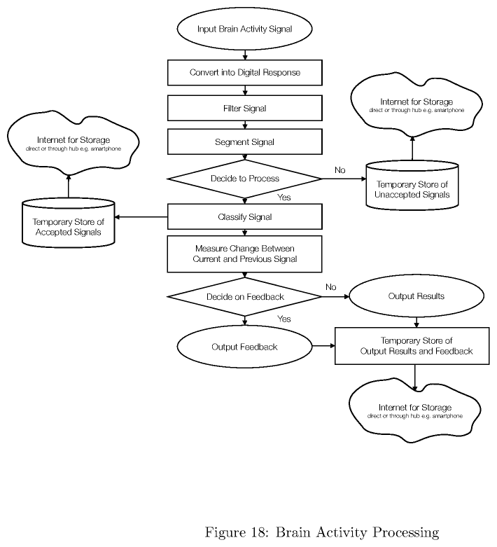

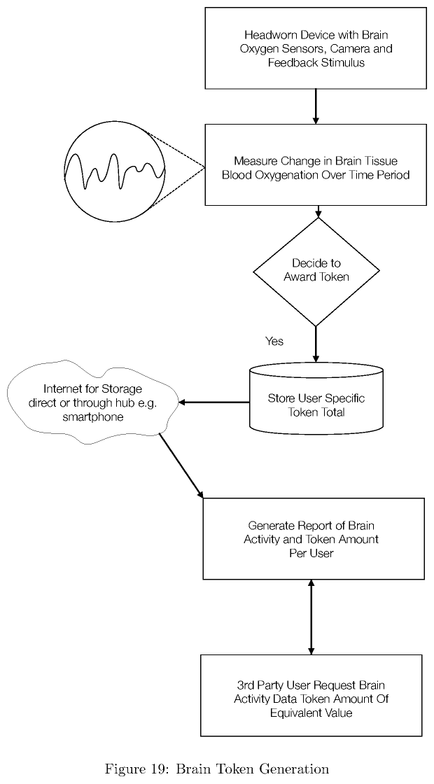

The embodiments description here within may be interpreted as a hardware and software combination. These parameters may be implemented on a computing hardware device consisting of at least one processor, one digital storage system of volatile and nonvolatile memory, and at least one communication interface. FIG. 15 outlines a novel low cost circuit that results in an output of measurement of light refraction from a light emitting diode as by a photodiode.

The processor which processes the digital output from the hemodynamic sensor may utilize a combination of a mathematical filtration library, an optimized classification library and a state decision logic library to output a classification and categorization on a fixed time interval as defined by the system.

In some embodiments on a human being a user may conduct a specific task and the sensor and computation system may read the output from the hemodynamic signals or series of signals to interpret automatically the activity being performed by the user and an activity level associated with the task classification.

According to the various embodiments a sensory system consisting of hemodynamic sensors may be applied in a variety of industries including sports, healthcare, safety, education and workplace monitoring.

An automatically generated model using a series of computational generated networks including general adversarial networks, recurrent neural networks, feed forward neural networks, convolutional neural networks and or random forest tree decisions based on filtration techniques outlined in [0025], are combined to automatically detect the activity state of the wearer and prediction a next activity state and score.

A series of filtration techniques include discrete wavelet transform, fourier transform on the third order cumulant (skewedness) and or a moving average, low pass, high pass and or a band pass filter.

Embodiments described herein provide a method and system to adjust the cognitive state of a mammal using a selection of feedback mechanisms

In particular the feedback mechanisms may include auditory, visual or haptic, electrical direct or alternating current feedback.

From the output of the cognitive model a feedback mechanism is selected by the wearer to adjust auditory, visual, haptic, electrical direct or alternating current feedback response.

The auditory feedback can include short form prose to give encouragement or suggest actions to the wearer.

The auditory feedback can include a short binaural pulsation across a 1 second to 90 seconds window of time

The visual feedback can include a light of specific color correlated with the cognitive state of the wearer and or an adaptive led or electronic tinting lense to adjust the light that filtering through a glasses lense as depicted in FIG. 9. The visual feedback can be presented as a visual stimulus encoded as described in FIG. 15 which outlines 1510 as the light emitting diode position, 1520 outlines the first LED color state for example green, followed by 1530 the time for which the color remains for example 1/15th of a second or 1/12th of a second before moving to state 1540 which results in a new color change for example cyan followed by a third color state and so on and is repeated. The states of colour and time between states can be continuous variation throughout the iterative loop.

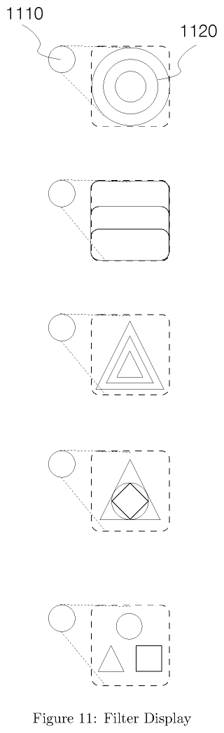

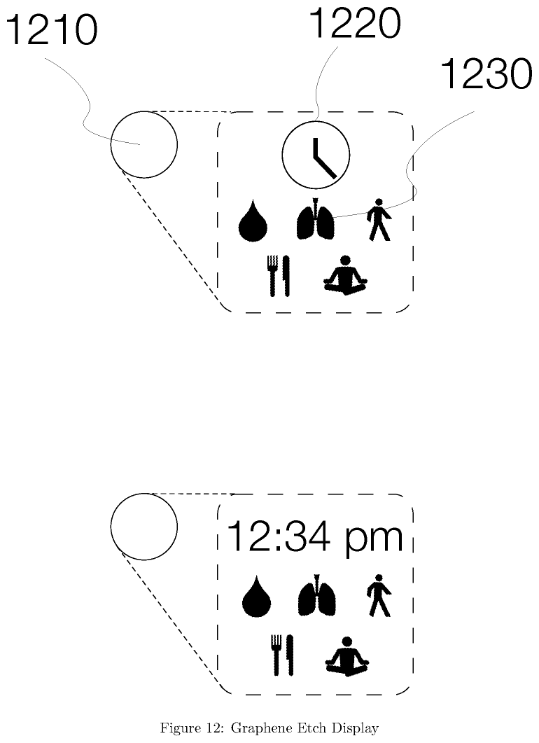

The visual feedback can include a light shined on to a semi reflective surface visible by the wearer for instance the top left corner of a glasses frame and or through a light channel as depicted in FIG. 10

The haptic feedback can include a short, long or multi-pulsation feedback to provide the wearer insight of current cognitive state

The feedback mechanism is triggered by a personalized threshold for feedback from the wearer based on a historical baseline on a range value from 0 to 100 percent cognitive level for the specific task the wearer is performing.

Task selection is determined by cognitive function and location of device including language, visual, and motor functions

Feedback is automatically determined based on a predefined user input selection

Audio or visual feedback in written form can include suggestions including short movement actions such as breathing, body position and or actions such as calling, messaging, closing, opening, drinking, walking, standing, sitting, laying, stretching and moving.

Embodiments described herein provide a method and system to provide a probability function of the cognitive state of a mammal using a selection of input including a hemodynamic response, time, a semantic input, and a cognitive state.

In particular the output mechanisms may include [symbol] cognitive state, probability [symbol] , [symbol] cognitive state as a real number from 0 to 1 [symbol] , [symbol] semantic string, probability [symbol] , [symbol] semantic string

[symbol]

In some embodiments on a human being a user may conduct a semantic output from a hemodynamic response measurement and generalized cognitive model.

From the output of the cognitive model and semantic model a closed loop feedback system can be inferred to provide a likelihood of semantic and or cognitive state output.

The semantic output can be provided in text and or auditory output.

While the teaching herein include illustrative embodiments and examples of some aspects of an invention, the description is not intended to be construed in a limiting sense. Thus, various modifications of the illustrative embodiments, as well as other embodiments of the invention, may be apparent to persons skilled in the art upon reference to this description. It is therefore contemplated that the appended claims will cover any such modifications or embodiments.

All publications, patents, and patent applications referred to herein are incorporated by reference in their entirety to the same extent as if each individual publication, patent, or patent application was specifically and individually indicated to be incorporated by reference in its entirety. From the foregoing description, it will thus be evident that the present invention provides a design for a human-machine interface. As various changes can be made in the above embodiments and operating methods without departing from the spirit or scope of the invention, it is intended that all matter contained in the above description or shown in the accompanying drawings should be interpreted as illustrative and not in a limiting sense.

Variations or modifications to the design and construction of this invention, within the scope of the invention, may occur to those skilled in the art upon reviewing the disclosure herein. Such variations or modifications, if within the spirit of this invention, are intended to be encompassed within the scope of any claims to patent protection issuing upon this invention.

What is claimed is:

1. A cybernetic human-machine interface, said interface including:
at least one wearable sensor to sense a mental state of a wearer of said user interface;
at least one feedback mechanism to provide real-time feedback and effect for the wearer
a processor for processing output from said sensor;
a transmitter for transmitting computational output and feedback to a secondary computational device
a receiver for receiving input from a secondary computation device
a computer implemented method for determining a value of change in oxygenated, de-oxygenated and total hemoglobin from a series of LEDs and photodiodes.
a computer implemented method for classification of hemodynamic signal or multiple hemodynamic signals to automatically interpret a users behaviour activity based on a predefined training model described as a flow profile

2. The method of claim 1, wherein the brain activity measurement is the resultant of a series of at least one photodiode and at least one light emitting diodes pairs with a series of signal generators to result in an analog measurement of brain activity from measured reflected light.

3. The method of claim 1, wherein the output results in a complex-valued signal representation of brain activity versus a reference brain activity

4. The method of claim 1, wherein the brain activity response is provided a rushing, dragging, leading, lagging feedback mechanism based on the wearer seeing visual content and or hearing audio content

5. The method of claim 1, wherein the output represents a leading, lagging, rushing and dragging complex-value

6. The method of claim 1, wherein the feedback is comprised of a combination of leading, lagging, rushing and dragging representation to the wearer

7. The method of claim 1, wherein the collective output of at least two wearers combines to modulate a visual and or audio representation of a complex-valued signal return of the collective

8. The method of claim 1, wherein the feedback mechanism is at least one of the following: an audio adjusted tone, a binaural tone, at least one light emitting diode, at least one pulse width modulation light emitting diode, at least one LED display, an alternating current, a direct current, a heat coil, a haptic pulsation

9. The method of claim 1, wherein the model comparison task comprises of at least one of: (i) a significant feature of data model event (ii) a unique series of features events (iii) a significant series of events

10. The method of claim 1, wherein the data event comprises of at least (i) one short task period for the user to be performing or analyzed (ii) an activity that occurs repeatedly over a period of time for activities including working, reading, listening, speaking, writing, thinking, meeting, conversation, programming, number work, other, gaming, watching, meditating, sleeping, running, jogging, walking, driving

11. The method of claim 1, wherein a closed loop methodology is applied to automatically provide a probability output including a cognitive state of a mammal and or a semantic input or output using a cognitive state model and hemodynamic response model or input

12. The method of claim 1, wherein the hemodynamic sensor is placed near or on the inferior frontal gyrus in the prefrontal cortex (FT7, FT8, F7, F8) and or left and or right temporal lobe (FT8, FT9, P9, P10, T7, T8) and or the brocca region (C5, C6, FC5, FC6), or the temporoparietal junction (TP7, TP8) from brain regional sections

13. The method of claim 5, wherein the cognitive model is adjusted based on the hemodynamic response, semantic likelihood and or a cognitive state baseline represented in a flow profile

14. The method of claim 1, wherein the output of the model results in a feedback mechanism that alters the cognitive state of the wearer

15. The method of claim 1, wherein the output of the model results in a feedback mechanism that alters the physical state of the wearer

16. The method of claim 1, wherein the output of the model results in a feedback mechanism that alters the cognitive state of another human

17. The method of claim 1, wherein the output of the model results in a feedback mechanism that alters the cognitive state of another machine

18. The method of claim 4, wherein the text input is provided by a human.

19. The method of claim 1 comprising of a plurality of datasets

20. The method of claim 1 comprising a flow profile which creates independent context by use of location, date, time and wearer descriptive data including hair thickness, face sizing parameters, skin tone, age, weight, height, percent body fat, skull thickness, absorption coefficient, extinction coefficient, differential pathlength factor

21. The method of claim 1 comprising of a re-trainable data model

22. The method of claim 1 wherein the previously trained model is a machine learned model

23. The method of claim 1 wherein the previously trained model is a statistical features

24. The method of claim 4 wherein the device is located in or on the frame of an eyeglass

25. The method of claim 4 wherein the device is a hair clip

26. The method of claim 4 wherein the device is located in a headband

27. The method of claim 4 wherein the device is located in a virtual reality headset

28. The method of claim 4 wherein the device is located in an augmented reality headset

29. The method of claim 1 wherein the device is located in or behind a pair of over the ear headphones

30. The method of claim 1 wherein the device is located in a hat

31. The method of claim 1 wherein the device is located in a hearing aid

32. The method of claim 1 wherein the device is located in a safety helmet

33. The method of claim 1 wherein the device is located in a necklace

34. The method of claim 1 wherein the device is located in a face mask

35. The method of claim 1 wherein the output “rushing”, “tempo”, “dragging” is used to control a camera and or lidar at variable frame rates wherein a slower frame rate is capture in a “dragging” state and a faster frame rate is captured in a “rushing” state

36. The method of claim 1 wherein the feedback mechanism applied function is generated through a chirplet transform or wavelet transform

37. The method of claim 1 where the output generates a exchangeable token whereby a 3rd party can exchange the token

38. The method of claim 1 where the output is serotonin generation, 5-HT, neurotransmit for impacting feelings

39. The method of claim 1 where the output is glutamate generation, GLU, sending signals to other cells

40. The method of claim 1 where the output is gamma-aminobutyric acid generation, GABA, reducing neuron excitability
